(19) United States
(12) Patent Application Publication
(10) Pub. No.: US 2009/0220931 A1
(43) Pub. Date: Sep. 3, 2009

(54) FUNCTIONAL IN VITRO IMMUNOASSAY

(75) Inventors: Manuel Schmidt, Berlin (DE); Burghardt Wittig, Berlin (DE); Astrid Sander, Berlin (DE); Yiyou Chen, San Jose, CA (US)

Correspondence Address: URSULA B. DAY, ESQ., 708 Third Avenue, SUITE 1501, NEW YORK, NY, 10017, US

(73) Assignee: Mologen AG, Berlin (DE)

(21) Appl. No.: 12/066,365

(22) PCT Filed: Sep. 8, 2006
(86) PCT No.: PCT/DE06/01604

§ 371 Date: Aug. 28, 2008

(30) Foreign Application Priority Data
Sep. 8, 2005 (DE) PCT/DE2005/001594
Oct. 26, 2005 (EP) 05090297.2

(51) Int. Cl. A01N 1/02; C12Q 1/02; C12Q 1/68; G01N 33/53

(52) U.S. Cl. 435/2; 435/29; 435/6; 435/7.24

(57) ABSTRACT

The invention relates to a method for the in vitro investigation of the effect of substances in in vivo processes and an in vitro detection method for the identification of immunomodulating compounds and/or the detection of the effect of immunomodulating compounds and the identification of apoptosis-inducing and/or necrosis-inducing compounds mediated by the immune system in in vivo processes. The methods according to the invention are particularly suitable for investigating effects of substances on cells, which are mediated by the immune system. Furthermore, the method according to the invention is suitable for in vitro monitoring of the in vivo effects before, during and/or after the administration of immunomodulating compounds and of apoptosis-inducing and/or necrosis-inducing compounds.

The invention relates to a method for the in vitro monitoring of the effect of substances in in vivo processes and to an in vitro detection method for identifying immunomodulating compounds and/or for detecting the effect of immunomodulating compounds as well as for identifying compounds which induce apoptosis and/or necrosis mediated by the immune system in in vivo processes.

In the pharmaceutical industry completely new classes of substances have been developed in recent years, which are intended for the therapy of the most varied diseases. These also include means from gene therapy or substances naturally occurring in the body that have been modified by gene therapy, such as for example proteins or DNA constructs.

Since there is as yet no experience with some of these completely new classes of substances in the pharmaceutical treatment of diseases, a need exists for methods of testing the effectiveness of these means, without having to fall back directly on animal experiments or clinical studies with patients. Such experiments using new, unknown substances are prohibited purely for ethical reasons. Instead, in preparation for this step, in vitro investigations are indicated to obtain results that allow statements concerning the in vivo effectiveness of the substances. Here it is essential in the in vitro experiments to come as close as possible to the in vivo situation.

Furthermore, it is important to develop simple methods for monitoring patients before, during and/or after a treatment method (e.g. immunotherapy or therapy that influences the immune system), whereby the reaction of the organism or the immune system is investigated in relation to the corresponding treatment method.

Alongside the conventional treatment methods for cancers such as radiotherapy and chemotherapy, which have represented the only treatment option for advanced cancers with metastases since the 1950s, it is now an objective to develop therapies that are associated with fewer side-effects for the patient, but which are highly effective in relation to achieving the goal of therapy.

One approach to this is immunotherapy, which aims to enhance the natural immune response to the cancer through genetically engineered modifications, that is, to influence the “attention” of the immune system vis-à-vis cancer cells and thus to influence the immune response so that the tumor is combated by the body itself.

Currently most clinical studies are based on the removal of the tumor, followed by ex-vivo transfection of the tumor cells with a therapeutic gene, radiation of the tumor cell population followed by reimplantation of the now modified tumor cells. This tumor cell vaccination allows the anti-tumor response to increase to varying degrees depending upon the transfected therapeutic gene.

In addition to the transfection of tumor cells, however, immunomodulating substances are also in development which are intended to induce the immune system to combat tumor cells. These immunomodulating substances are intended to induce or “program” the immune system so that tumor cells are specifically attacked and ultimately destroyed. In this approach, immunomodulating substances in cancer therapy act indirectly via the immune system on the relevant tumor or the underlying type of tumor cell.

A method that allows the in vitro investigation of the effect of new substances on in vivo processes, for example the destruction of tumor cells, would on the one hand avoid in vivo experiments subject to major ethical reservations, and on the other hand would make it possible to test a large number of substances with a large number of different tumor cells in a short time. Furthermore, with such a method it would be possible to show the progress of a therapy in relation to the induced in vivo effects in so-called “therapy monitoring.”

In view of this state of the art the task of the present invention is to provide a method that allows in vitro investigation of the effectiveness of substances on in vivo processes in humans or higher mammals.

This task is fulfilled by the features of the independent claims.

In the sense of the invention:

| | |
|---|---|
| Effector cells | means a mixture of immune cells, such as e.g. PBMC |
| of the immune | [peripheral blood mononuclear cells (from humans |
| system | or higher mammals), spleen cells (animal models), |
| | etc.] or subpopulations sorted by FACS or MACS, |
| | e.g. B, T and NK cells, monocytes, dendritic cells, etc. |
| CpG motif | means unmethylated cytosine guanine motif |
| dSLIM | means double stem loop immunomodulating |
| | oligodeoxyribonucleotides, whereby every loop |
| | exhibits CpG motifs, preferably three |
| ODN | means oligodeoxyribonucleotide |
| PBMC | means peripheral mononuclear blood cells |

A number of general concepts are to be understood below as follows:

Immunomodulating compounds in the sense of the present invention are to be understood as substances that are able to influence the reaction of the immune system, or only individual cells thereof, in particular the effector cells. Alongside chemical compounds these include also DNA constructs, proteins, antibodies, sugar molecules or other substances which exhibit the properties that lead to the immune system or cells of the immune system being caused to react. This relates in particular to the cells of the immune system that are termed effector cells in the present invention, which are able to effect or mediate reactions of the immune system. This mediation takes place via the release of specific messenger substances.

Accordingly, the invention relates to a method which comprises the following method steps:
a) isolation of cells
b) primary incubation of the cells with the substance to be investigated
c) recovery of the supernatant or of the mixture of cells and supernatant from the primary incubation
d) secondary incubation of target cells with the supernatant or the mixture of cells and supernatant
e) analysis of the target cells.

An alternative embodiment relates to an in vitro detection method envisaged for the identification of immunomodulating compounds and/or the detection of the effect of immunomodulating compounds and the identification of apoptosis-inducing and/or necrosis-inducing compounds mediated by the immune system in in vivo processes, which comprises the following sequence of steps:

a) primary incubation of effector cells of the immune system with an apoptosis-inducing and/or necrosis-inducing substance that is to be investigated for immunomodulating effect, followed by the

b) recovery of the supernatant or of the mixture of cells and supernatant from the primary incubation and the following

c) secondary incubation of target cells with the supernatant or the mixture of cells and supernatant from the primary incubation, and finally

d) the immunomodulating and/or apoptosis-inducing and/or necrosis-inducing effect is analyzed by means of a suitable detection method.

The steps in the method indicated make it possible to investigate in vitro the effect of substances in in vivo processes. As a result, new types of compounds can be tested under conditions that come very close to those in the in vivo situation, without endangering animals and/or patients in clinical studies.

Furthermore, the impact of a therapy already planned/carried out can be monitored (by the analysis of relevant parameters). This use of the method according to the invention is also termed “therapy monitoring” in the sense of this invention. This term is applied solely to the in vitro monitoring of the in vivo therapeutic effects. The methods according to the invention are not themselves connected with the therapy, except that the success of the therapy can be monitored.

The isolated cells are effector cells of the immune system in accordance with the above definition in a preferred embodiment of the method according to the invention. The methods according to the invention are particularly suitable for investigating effects of substances on cells which are mediated by the immune system.

After cells of the immune system together with the substances in the primary incubation were able to exert their effect on the latter, in the secondary incubation the in vivo effects of the substance were then shown by incubating the supernatants or the mixture of cells and supernatant from the primary incubation, which contain amongst other things the secreted products of the cells of the immune system, with target cells.

Preferred target cells are to be human cells or cells from higher mammals. In a particularly preferred embodiment of the methods according to the invention, isolated cells are used for the primary incubation, in particular cells of the immune system, and as target cells for the secondary incubation either tumor cells or cell lines genetically descended from tumor cells. In this embodiment of the method according to the invention, the latter is then termed “Functional in vitro immunoassay.”

In principle any types of tumor cells of differing origin can be considered as tumor cells. The objective of a “functional in vitro immunoassay” is to identify or investigate substances that are suitable for initiating apoptosis or necrosis in tumor cells through the immune system.

However, another objective of the methods according to the invention is to investigate the recognition of tumor cells by the immune system, triggered by the enhanced expression of MHC-I (e.g. HLA-ABC) and adhesion molecules (e.g. ICAM-1) on the surface of the tumor cells. A decisive advantage of the methods according to the invention is that the in vivo effect can be detected without the need to conduct experiments in animals and/or patients in clinical studies, with all the associated disadvantages.

A kit is provided according to the invention for application of the methods according to the invention for the investigation of changes in the expression of surface molecules owing to an immune reaction induced by the immunomodulating substance. The kit contains aliquots of cells prepared for storage, preferably effector cells of the immune system, for the primary incubation with the substances to be investigated, means of carrying out primary and secondary incubation and suitable means of analysis of the expression pattern of the surface molecules of the cells from the secondary incubation. For analysis of the expression pattern of surface antigens of the target cells of the secondary incubation, the kit according to the invention contains means of carrying out an RT-PCR, whereby the kit contains suitable primers for multiplication of the mRNA from surface molecules, enzymes for multiplication and the required buffers and/or means of FACS analysis, for which the kit contains suitable fluorescence marked antibodies that are directed against surface antigens and apoptosis/necrosis markers and, in addition, means of preparing the target cells, such as buffers and chemicals.

In a further development the methods according to the invention are also suitable for therapy monitoring, whereby whole blood, blood cells, blood serum or the blood plasma of a patient is used as the substance to be investigated in the primary incubation before, during and/or after a treatment (e.g. immunotherapy or therapy that alters or influences the immune system).

By means of this further development of the methods according to the invention, it is possible to examine whether therapeutic agents that were administered to the patient and preferably have a stimulating action on the immune system, have already produced an in vivo effect. Although in the method the blood of the patient is investigated with the cells contained therein and/or messenger substances or parts thereof (e.g. serum and/or plasma or cell subpopulations), in this embodiment a method according to the invention ultimately serves the indirect detection of the in vivo effect of the substance which was administered to the patient in the therapy, preferably an immunotherapy.

If no specific antibodies are known that can be used for “therapy monitoring” in the methods according to the invention, it is possible to monitor an in vivo effect via changes in the cytokine level in the blood (plasma/serum), or changes in the production of specific antibodies following a reaction of the immune system, after the administration of therapeutic agents.

The treatments in which the methods according to the invention are provided as therapy monitoring of the effectiveness of the therapeutic agents used in each case, are preferably for diseases such as cancer, infections, allergies and autoimmune diseases.

Due to the advantages mentioned, therefore, compounds are also preferably envisaged for the methods according to the invention which have an immunomodulating effect or are able to induce apoptosis or necrosis.

According to the invention CpG-motif-containing oligodeoxynucleotides and dSLIM (double stem loop immunomodulating oligodeoxyribonucleotides, see EP 1 196 178 B1) are preferably envisaged as immunomodulating compounds. However, within the scope of the invention other biomolecules may also be used, such as for example natural or genetically modified antibodies, DNA-based and/or RNA-based substances (antisense oligodeoxynucleotides, si-RNA, etc.), amino acid compounds, messenger substances or other immunomodulators (such as for example aluminum salts, imidazoquinolines, lipopolysaccharides, saponin derivatives, phospholipids, squalenes, etc.).

According to the invention, in particular those compounds can be considered as apoptosis-inducing and/or necrosis-inducing compounds that are suitable for permanently disrupting the processes necessary for maintenance of the cells. Here in particular DNA-based and/or RNA-based substances (antisense oligodeoxynucleotides, si-RNA, etc.), antibodies or chemotherapeutic agents can be considered.

Furthermore, the methods according to the invention can be used to identify messenger substances that are released by the cells following the incubation of the isolated cells in the primary incubation with immunomodulating or apoptosis-inducing and/or necrosis-inducing substances. For this, before being added to the target cells of the secondary incubation, the supernatant from the primary incubation is pre-incubated with antibodies that specifically recognize potential messenger substances. The interaction between the antibody and epitope of the messenger substance renders the latter unable to send signals to the target cells and in this way its function is blocked. This embodiment of the method according to the invention is important for detecting which specific messenger substances are responsible for an induced effect, e.g. apoptosis.

Multi-well plates with 24 to 96 wells are preferably used in a kit for application of the methods according to the invention for identification of the induced release of messenger substances, whereby the surface of each well of a plate is coated with an antibody that is directed against an epitope of a messenger substance (e.g. IFN-γ) and after incubation of fractions of the supernatant from the primary incubation with a plate pre-treated in this manner and the following incubation of the fractions with target cells, there is the possibility of testing a large number of potential messenger substances within a short time to find out whether they are in fact involved in the mediation of an immune response or the induction of apoptosis.

The invention thus also relates to a kit for application of the methods according to the invention for the identification of messenger substances that are released as a reaction of the incubation of the cells in the primary incubation with a substance to be investigated. A kit of this type contains aliquots of cells prepared for storage, preferably effector cells of the immune system, for the primary incubation with the substances to be investigated, means of conducting primary and secondary incubation, and in addition multi-well plates with 24 to 96 wells, in which the surfaces of the wells are coated with an antibody, whereby the surfaces of various different wells are coated with different antibodies, preferably however, at least two wells each with an identical antibody.

The necessary incubation steps in the methods according to the invention take place preferably in an incubator containing 5% CO₂. However, other incubation conditions are also conceivable that are adapted to the requirements of the cells to be incubated in each case.

The recovery of the supernatants or of the mixture of the supernatant and the cells from the primary incubation takes place according to the invention by centrifugation. However, according to the invention also all other methods are conceivable that are suitable for separating the cells from the supernatants, such as for example filtration of the cells with a pore size that allows only the supernatant to pass but not the cells or any cell debris present. Furthermore, cell separation systems and/or cell sorting systems using specific antibodies followed by magnetic (MACS) or fluorescence-based (FACS) selection are envisaged.

For the analysis of the cells according to the invention methods are envisaged that can show changes to the protein expression in the target cells. Here FACS measurements (fluorescent activated cell sorting), Western blots, gel filtration or cytospins can be considered in particular.

Furthermore, methods for analysis of changes in the expression of certain genes are envisaged, such as for example RT-PCR, real-time PCR, RNase protection assays and Northern and Southern blots.

Finally in the analysis of the in vivo effects apoptosis assays are also envisaged, such as for example staining of the cells with annexin V or the TUNEL assay, or cell cycle analyses, e.g. by means of propidium iodide staining.

The examples and results of experiments listed below demonstrate that the application of a method according to the invention is not only able to represent using in vitro investigations the effect of substances in in vitro processes, but rather is also suitable for testing and documenting the specificity of the effects found by expanding a method according to the invention into a competition assay.

Further advantageous embodiments of the invention result from the dependent claims and the description. The invention, including the practicability of the method according to the invention, is described below in more detail using the examples of embodiments and figures, however without restricting the invention to these examples.

### Recovery of Mononuclear Cells

For carrying out the method according to the invention, peripheral blood mononuclear cells (PBMC) were extracted from either whole blood or what is called the “buffy coat.” This is a by-product that arises during the production of erythrocyte concentrates from whole blood.

The PBMC were isolated by centrifugation using a Ficoll gradient in order to separate erythrocytes, granulocytes and dead cells. Ficoll is an uncharged sucrose polymer whose density is set such that when it is covered with whole blood or buffy coat and then centrifuged, the fractions of lower density pass through the ficoll layer and collect at the bottom, while lymphocytes and monocytes collect in the interphase between the plasma (above) and the Ficoll (below).

The interphase, which contains the cells after centrifugation, was isolated and washed several times with PBS. Following this the isolated cells were taken up in cell culture medium and adjusted to a concentration of 1-4×10⁶ cells per milliliter.

Double Stem Loop Immunomodulating Oligodeoxyribonucleotides (dSLIM)

Double stem loop immunomodulating oligodeoxyribonucleotides are molecules with CpG sequences. They are obtained by closing linear oligodeoxynucleotides (ODNs) covalently by means of a nucleotide loop, so that they are protected against degradation by exonucleases. Thus dumbbell-shaped molecules are obtained, called dSLIM, “double stem loop immunomodulators.” Their immunomodulating activity is based on a nonspecific activation of the immune system by the non-methylated CpG sequences that bind to Toll-like receptors, and above all the special structure of the dSLIM molecules. Each loop of the dSLIM contains three non-methylated CpG motifs.

Double-stranded loop immunomodulators (dSLIM) of the ISS30 type (e.g. dSLIM-30L1) were synthesized according to SOP with subsequent quality control in a class B laboratory. For this, single-stranded hairpin-shaped 5′-phosphorylated oligodeoxyribonucleotides (ODN) were ligated with T4 DNA ligase. After digestion of the remaining starting materials with T7 DNA polymerase and chromatographic purification, the resulting dSLIM were concentrated by ethanol/sodium magnesium acetate precipitation and dissolved in PBS. The exact procedure is given in WO 01/07055.

Primary Incubation of the Immune Cells (PBMC) with dSLIM

The isolated cells (PBMC) were seeded out in multi-well plates. The size of the batches and, accordingly, the size of the wells, were selected so that the culture supernatant harvested later had precisely the volume that was required for the secondary incubation with the target cells.

A first batch contained unstimulated cells (negative control). A second batch was stimulated with 0.1-10 μM dSLIM-30L1. In two further batches cells were stimulated with 0.1-10 μM of an oligodeoxynucleotide (ODN) to give the strongest possible positive result, to allow the calibration of the devices and compensation in the FACS. In further batches cells were stimulated with 0.1-10 μM of other ODNs for comparison. Each batch was incubated for 48 hours in a CO₂ incubator at 37 degrees Celsius. The supernatants of these batches were recovered by centrifugation and frozen at −80 degrees Celsius for further work.

Secondary Incubation with Target Cells (e.g. HT-29)

For the secondary incubation with the target cells, the optimum concentration and the volume had to be determined in advance at which the target cells were seeded out. The objective was that after the secondary incubation at least 5×10⁵ target cells per well are available for the analysis. Here it had to be ensured that the cells had optimum growth conditions for three days and were seeded out as densely as necessary and as sparsely as possible, so that after three days they were almost confluent. Non-optimum growth conditions also lead to necrosis or apoptosis, which would corrupt the experimental result. In this case HT-29 colon carcinoma cells were used as target cells.

The cells were seeded out at the previously determined optimum density in batches of the corresponding size and incubated overnight in the CO₂ incubator at 37 degrees Celsius (e.g. 2.4×10⁵ cells in 700 μl per well in a 24-well plate).

Stimulation occurred on the next day by removal of the medium from the now adherent cells and addition of the supernatants from the primary incubation (“indirect stimulation”) or the substances indicated (dSLIM-30L1, lin30L1) directly to the medium (“direct stimulation”). As a negative control medium only was added to an indirect batch. These cells were termed untreated cells to distinguish them from the unstimulated cells (addition of unstimulated supernatant from primary incubation).

The batches—direct stimulation and indirect stimulation—were once again incubated for 48 hours in the CO₂ incubator at 37 degrees Celsius. After this, the analysis desired in each case could be carried out on the cells. For this firstly the supernatants were removed from the cells and the cells were washed with PBS. The cells were removed from the wells using trypsin/EDTA and after a further washing step they were transferred to a centrifugation tube for the following determination of the number of cells.

### Staining of Surface Antigens

The cells from the stimulation batches were centrifuged out and washed with a special staining buffer. After this the cell suspension was adjusted to a concentration of 1×10⁶ cells per milliliter. 500 μl (0.5×10⁶ cells) of this cell suspension was centrifuged off in a FACS tube and after being taken up in 50 μl of staining buffer the antibodies were added (e.g. ICAM-1 (CD54) conjugated with FITC, and HLA-ABC conjugated with PE). For each antibody a corresponding isotype control was provided, as was an individually stained positive sample for device calibration and compensation. After an incubation step the cells were washed twice with PBS and resuspended for the measurement in 500-1000 μl PBS. To distinguish the dead cells, 7-AAD was added and incubated for another 10 minutes. The FACS measurement then followed.

### Staining of Apoptotic/Necrotic Cells

Apoptotic cells were stained with annexin V-PE, which indicates apoptotic processes in the cells. Counterstaining with 7-AAD was performed to distinguish these cells from necrotic cells.

The cells from the stimulation batches were centrifuged off and washed twice with PBS. After this the cells were diluted in a special annexin binding buffer and adjusted to a cell concentration of 1×10⁶ cells per milliliter. 5 μl annexin V-PE and 7-AAD was added per 100 μl (1×10⁵ cells) of this cell suspension, and after thorough mixing this was incubated at room temperature for 15 min. Then 400 μl of binding buffer was added and the FACS measurement took place immediately.

Flow Cytometric Measurement with FACS

### Apoptosis/Necrosis

Fluorescence 2 (annexin V-PE) and fluorescence 3 (7-AAD) were measured. The devices were calibrated using unstimulated cells (direct batches) and/or untreated cells (indirect batches).

In the dot plot of FSC (forward scatter=cell size) against SSC (side scatter=cell granularity), the cell population was adjusted so that it was in the center. There followed the PMT calibrations and compensation for fluorescence 2 and fluorescence 3. After this all the samples were measured (5000 cells).

### B. Surface Antigens

Fluorescence 1 (ICAM 1-FITC), fluorescence 2 (HLA-ABC-PE) and fluorescence 3 (7-MD) were measured.

The devices were calibrated using cells stimulated by lin-30L1 with corresponding isotype controls (with double staining) for comparison of nonspecific binding and with the fluorescence marked antibodies (with single staining).

In the dot plot of FSC against SSC the cell population was adjusted so that it was in the center. There followed PMT calibrations for fluorescence 1, 2 and 3 using the isotype controls, and the compensation with single staining. After this all the samples were measured (10000 cells). Here the dead cells (7-MD positive cells) were excluded (fluorescence 3 versus FSC in the dot plot).

### Interpretation of Results

### Apoptosis/Necrosis

A dot plot was created showing 7-AAD versus annexin V. Then quadrants were drawn up based on untreated cells. Depending on the cells' position in the respective quadrants, they belong either to the apoptotic or the necrotic fraction.

living cells are annexin-negative and 7AAD-negative

(LL Quadrant)

apoptotic cells are annexin-positive and 7AAD-negative

(LR Quadrant)

necrotic cells are annexin-positive and 7AAD-positive

(UR Quadrant)

or

annexin-negative and 7-AAD-positive

(UL Quadrant)

B. Surface markers

Two dot plots (fluorescence 1 versus FSC, and fluorescence 2 versus FSC) were created with the living cells. The fluorescence intensity (fluorescence 1/ICAM-1 or 2/HLA-ABC) of the cells was read off depending on the cells' position in the respective dot plots. Then a comparison was made with the relevant controls.

Comparison of the test batch with the controls in relation to

number of surface-marker-positive cells (=number of cells with corresponding surface marker)

fluorescence intensity of the surface markers (=number of the surface marker molecules on the cell surface)

The results from carrying out the examples described using the method according to the invention are shown in the figures.

The figures show the following:

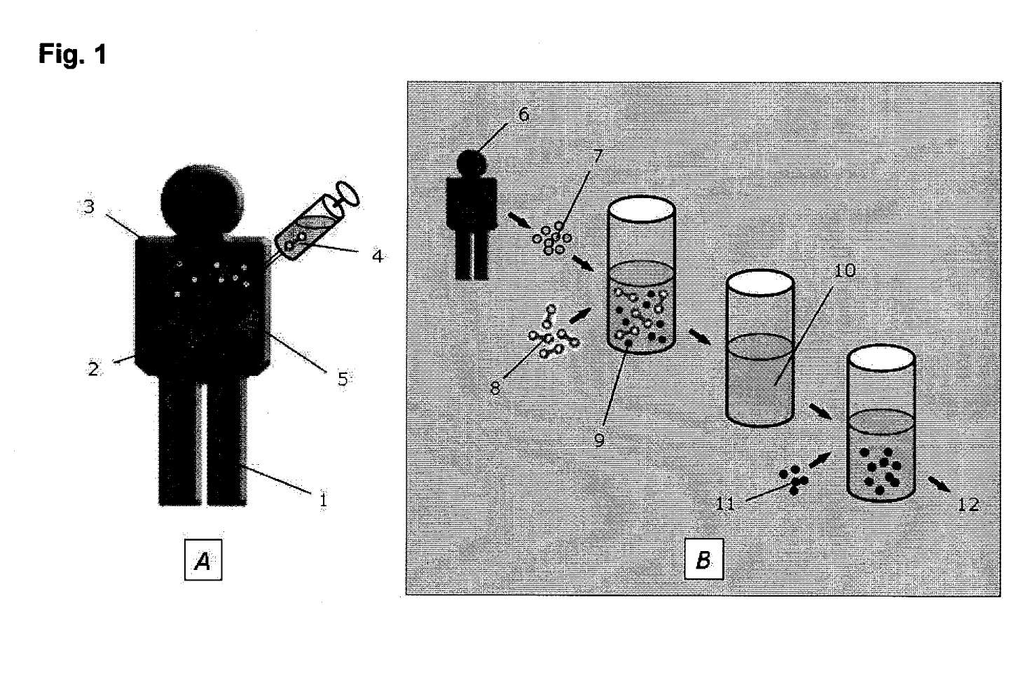

FIG. 1 Schematic representation of the method according to the invention.

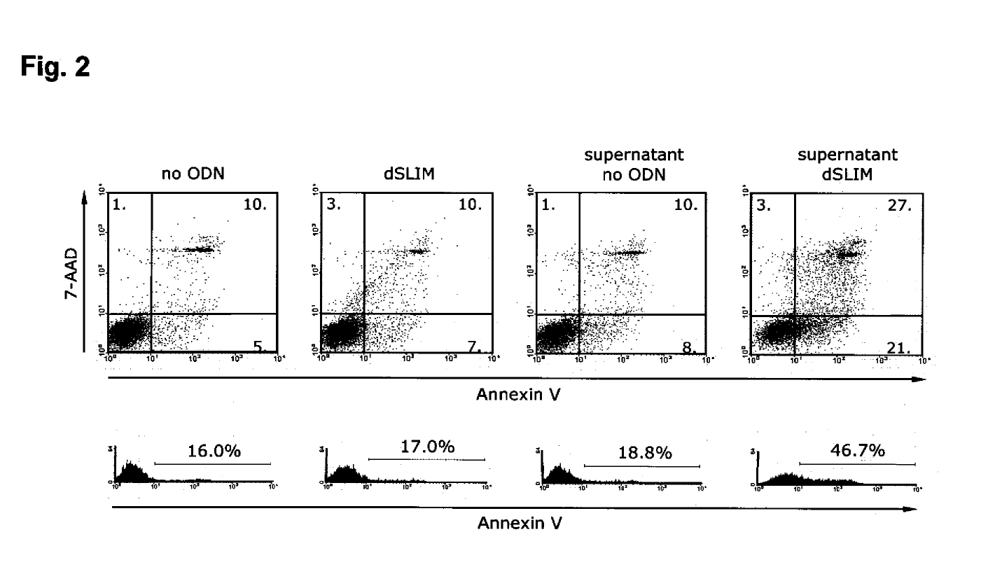

FIG. 2 Analysis of the in vitro effect of the dSLIM immunomodulator by detection of apoptosis and necrosis in HT-29 tumor cells.

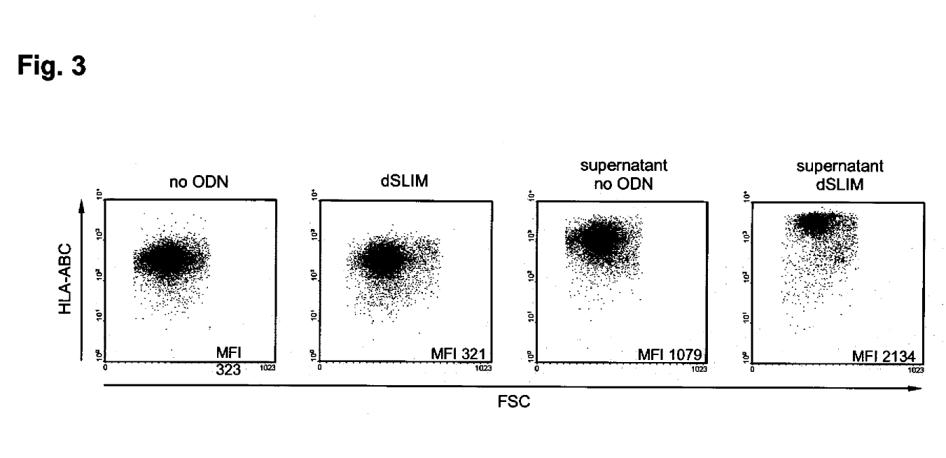

FIG. 3 Analysis of the in vitro effect of the dSLIM immunomodulator by detection of the expression of HLA-ABC surface markers in HT-29 tumor cells.

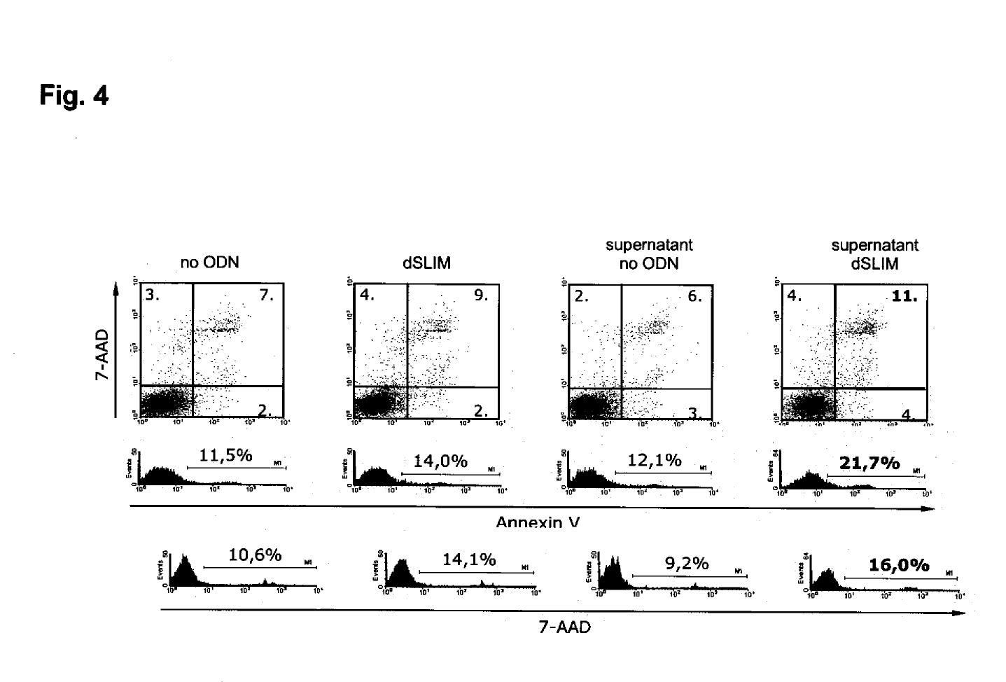

FIG. 4 Analysis of the in vitro effect of the dSLIM immunomodulator by detection of apoptosis and necrosis in HEK-293 tumor cells.

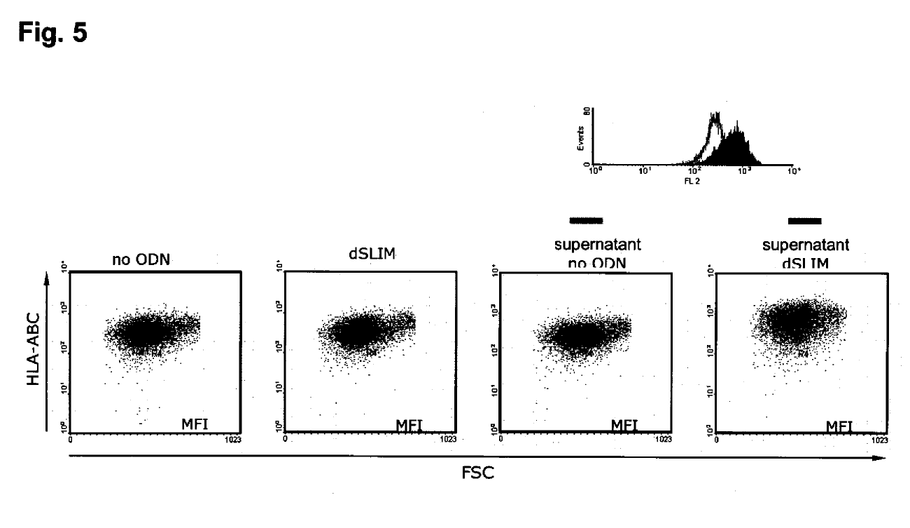

FIG. 5 Analysis of the in vitro effect of the dSLIM immunomodulator by detection of the expression of HLA-ABC surface markers in HEK-293 tumor cells.

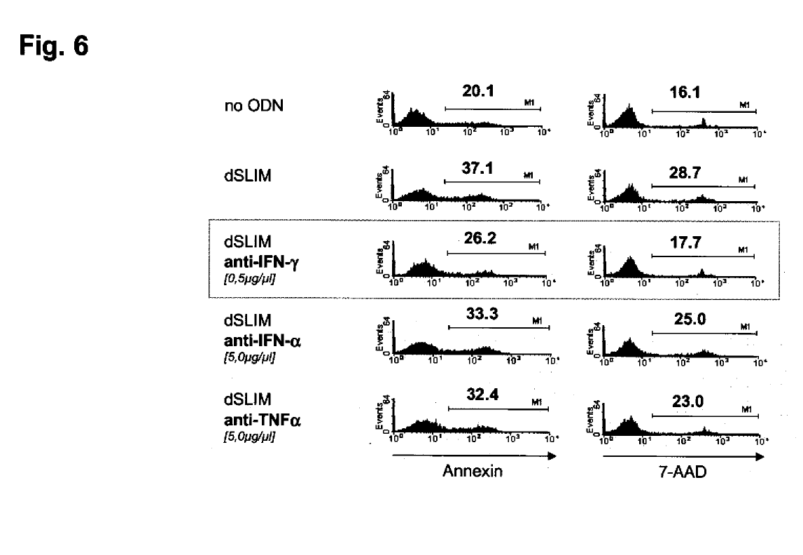

FIG. 6 Analysis of the mechanism of action of dSLIM by detection of apoptosis and necrosis in HT-29 tumor cells using the method according to the invention.

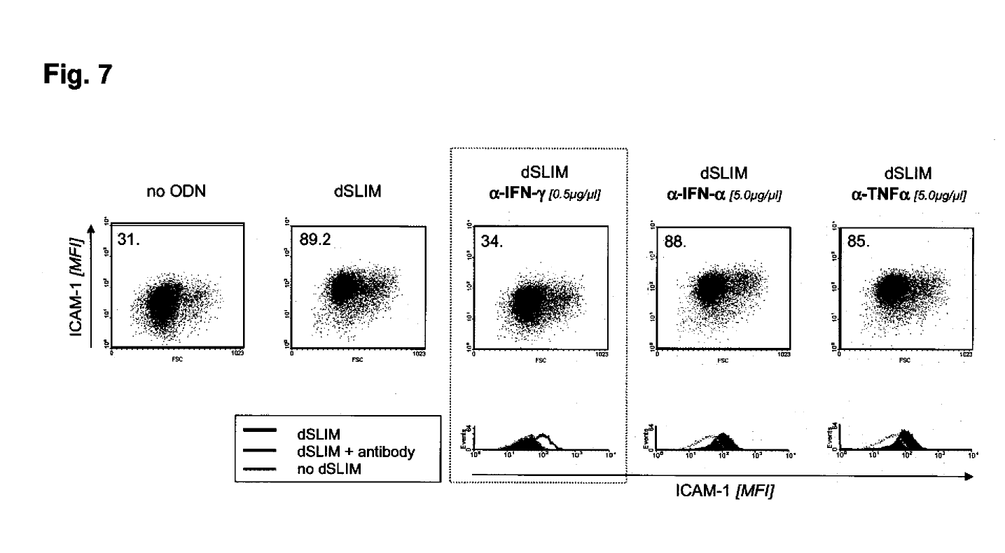

FIG. 7 Analysis of the mechanism of action of dSLIM by detection of the expression of HLA-ABC surface markers in HT-29 tumor cells using the method according to the invention.

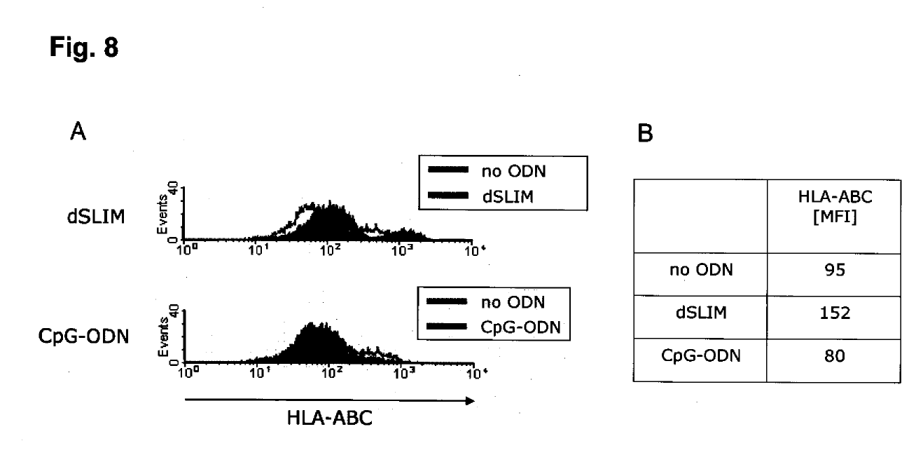

FIG. 8 Comparison of the effectiveness of dSLIM with linear CpG ODNs by detection of the expression of HLA-ABC surface markers in RENCA tumor cells.

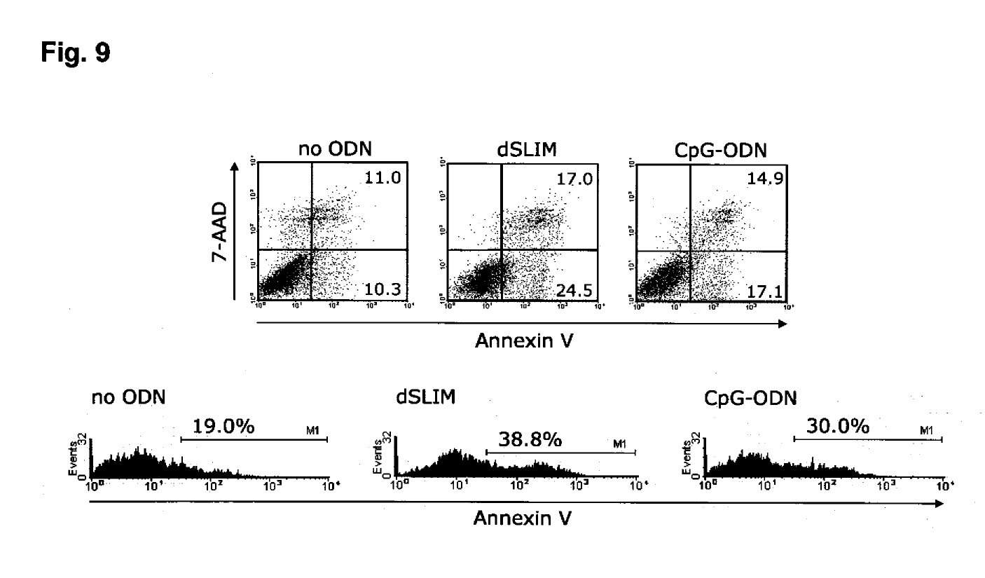

FIG. 9 Comparison of the effectiveness of dSLIM with linear CpG ODNs by detection of apoptosis and necrosis in RENCA tumor cells.

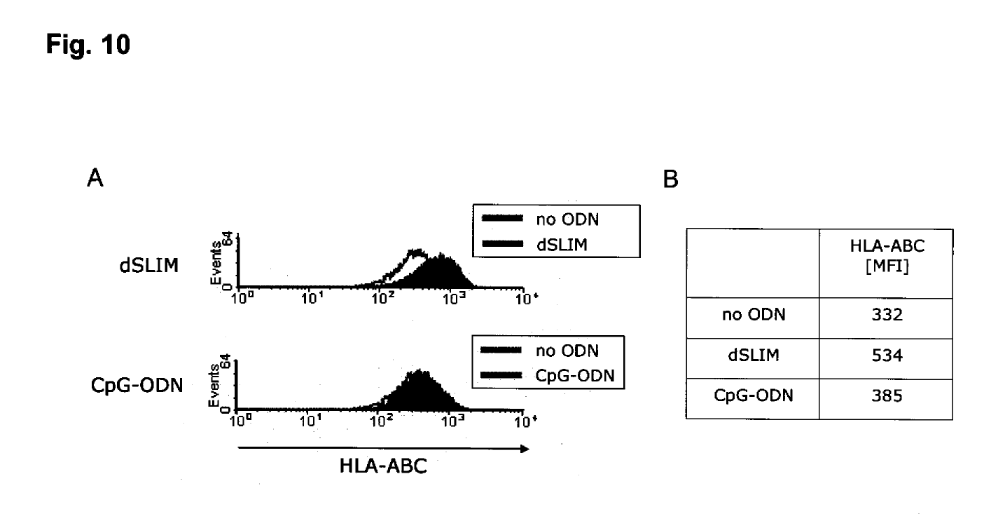

FIG. 10 Comparison of the effectiveness of dSLIM with linear CpG ODNs by detection of the expression of HLA-ABC surface markers in HT-29 tumor cells.

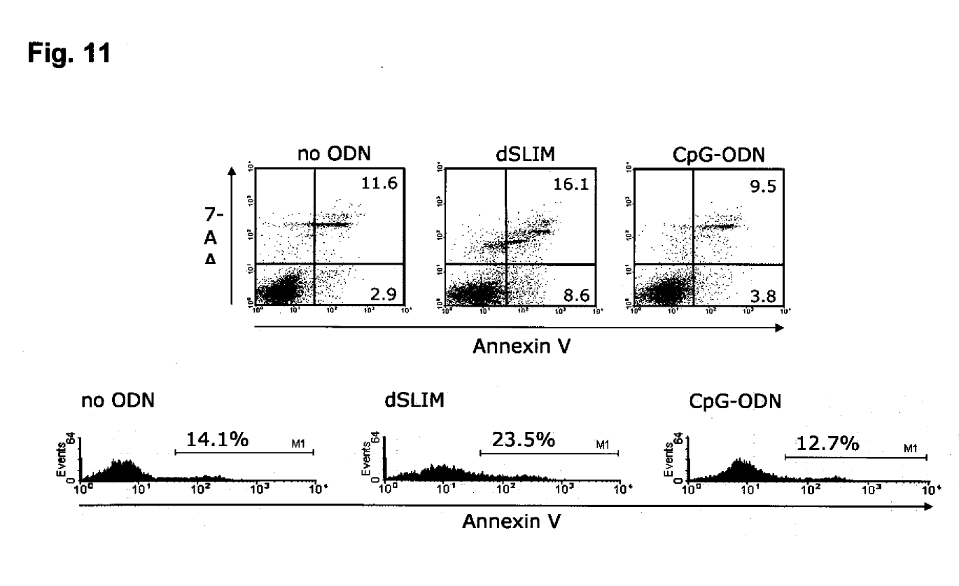

FIG. 11 Comparison of the effectiveness of dSLIM with linear CpG ODNs by detection of apoptosis and necrosis in HT-29 tumor cells.

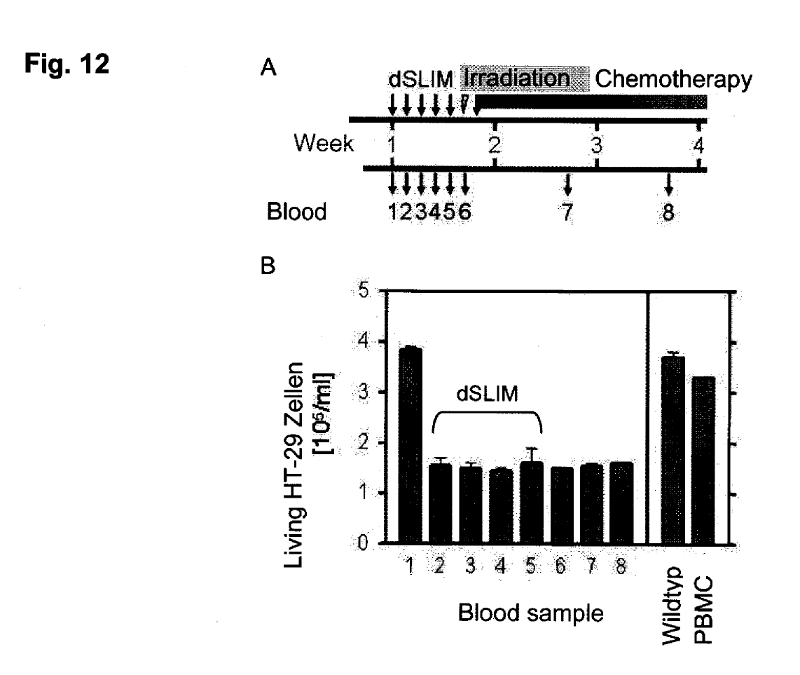

FIG. 12 In vitro monitoring of viable tumor cells during the therapy of a cancer patient.

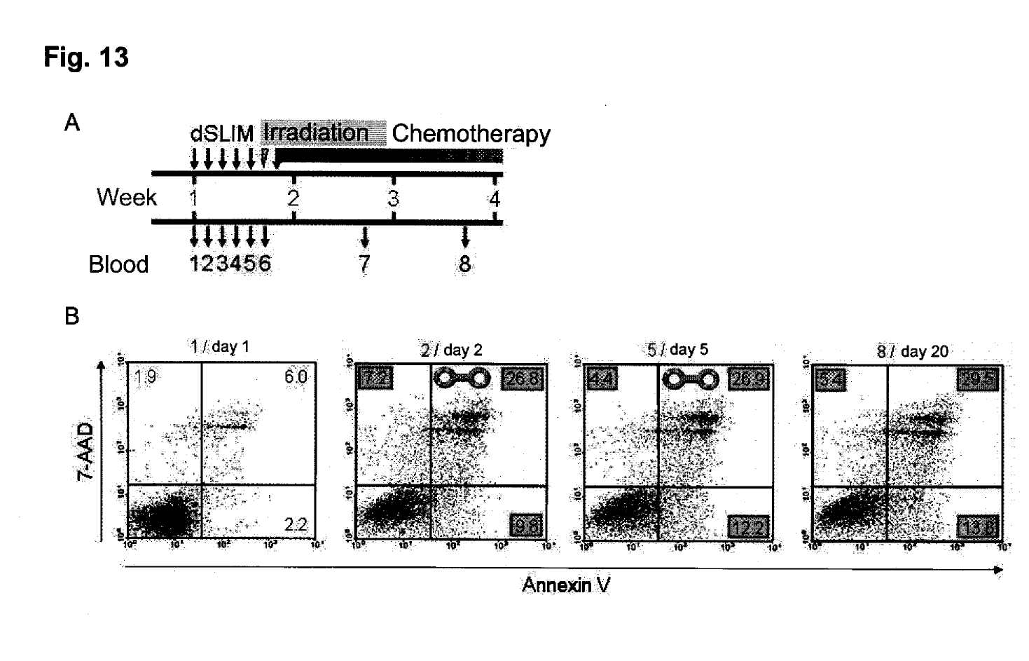

FIG. 13 In vitro monitoring of apoptotic/necrotic tumor cells during the therapy of a cancer patient.

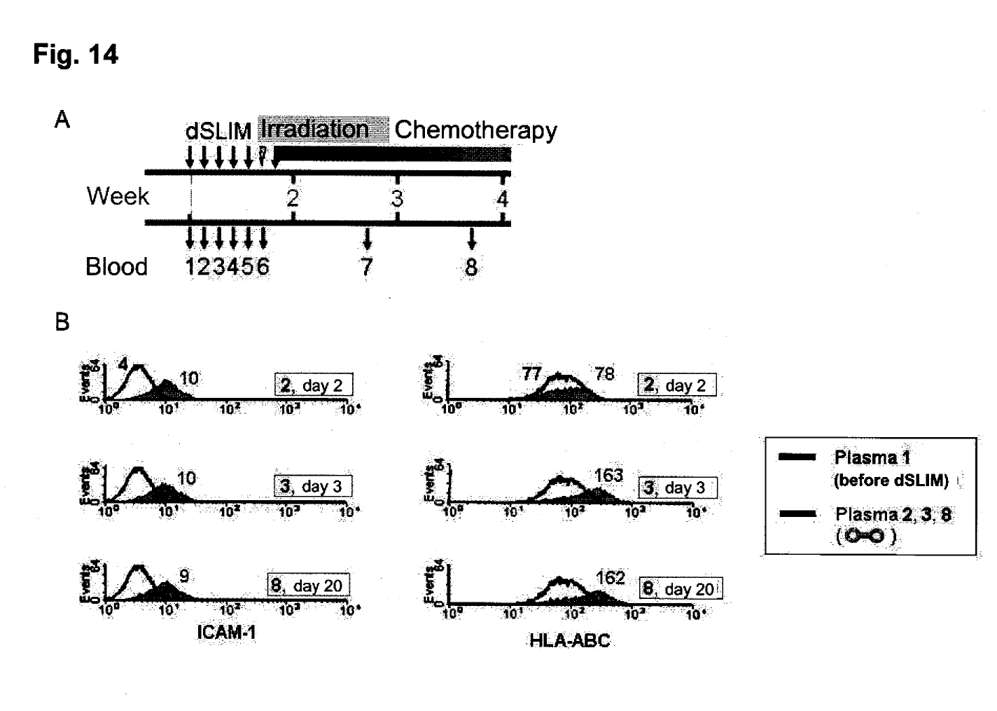

FIG. 14 In vitro monitoring of the surface markers of tumor cells during the therapy of a cancer patient.

FIG. 1 shows a schematic diagram of the sequence of the steps in the method according to the invention. Part A, on the left, depicts a typical application in vivo; part B, on the right, shows the relevant method according to the invention in the embodiment as “functional in vitro immunoassay.”

FIG. 2 shows the results of an analysis of the in vitro effect of the dSLIM immunomodulator applying the method according to the invention. The use of the supernatant from PBMCs incubated with dSLIM induces apoptosis and necrosis in HT-29 tumor cells (carcinoma of the colon), as can be seen in the right part of the figure. Here an increase in apoptosis can be seen from cells treated directly with dSLIM to the cells treated with the supernatant, from 17% to 46.7%.

In FIG. 3 the in vitro effect of the dSLIM immunomodulator in HT-29 cells is analyzed. The use of the supernatant from PBMCs incubated with dSLIM induces enhanced expression of HLA-ABC surface markers. The shift of the cell population can be recognized in the far right of the figure.

To back up the experimental results obtained in HT-29 applying the method according to the invention, analogous experiments were carried out in HEK-293 cells. The results are shown in FIGS. 4 and 5.

FIG. 4 shows that dSLIM induces apoptosis (annexin V) and necrosis (7-AAD). So the number of apoptotic cells rises due to the supernatant from the cells treated with dSLIM in comparison to the cells treated with a supernatant without ODN, from 12.1% to 21.7%. The number of necrotic cells rises from 9.2% to 16%.

FIG. 5 shows the enhanced induction of the HLA-ABC surface markers by the incubation of the target cells (HT-29) with the dSLIM supernatant from the PBMC. The upper part of the figure shows the shift (=increase in expression) of the population of cells that were treated with supernatant originating from PBMCs that were not treated with ODN, to cells that were incubated with the supernatant from the PBMCs treated with dSLIM.

FIG. 6 shows the results of an analysis of the mechanism of action of dSLIM in HT-29 cells applying the method according to the invention, and the detection of apoptosis and necrosis. Here in the step of primary incubation of the PBMCs, an antibody is added (anti-IFN-γ, green frame) that is able to neutralize the effect of dSLIM. For comparison, experiments with antibodies (anti-IFN-α, anti-TNFa) were carried out to prove the specificity. It can easily be seen (green frame) that the anti-IFN-γ antibody minimizes the number both of apoptotic cells and of necrotic cells.

In FIG. 7 the application of the method according to the invention corresponds to that in FIG. 6, but the expression of the surface marker ICAM-1 (CD54) on the target cells (HT-29) is analyzed. The shift of the cell population is shown for comparison in the lower part of the figure.

FIGS. 8 and 9 show results from experiments applying the method according to the invention in RENCA tumor cells, whereby the effect of dSLIM with linear ODNs was investigated for comparison. However, the linear oligodeoxynucleotides containing CpG also have a different sequence than the dSLIM and are protected by phosphorothioate against decomposition.

FIG. 8 shows that the treatment of the target cells with dSLIM leads to enhanced expression of the surface marker HLA-ABC (upper section), whereas a linear CpG ODN has no effect. The table on the right of the figure shows the numerical differences. As shown in FIG. 9, dSLIM is clearly more potent than linear CpG ODN in the induction of apoptosis and necrosis. The difference in the induction of apoptosis is indicated in percentages in the lower section.

FIGS. 10 and 11 compare dSLIM with linear CpG ODNs, applying the method according to the invention, in HT-29 cells as target cells. The results of these experiments correspond to the results that were obtained with the RENCA tumor cells and are shown in FIGS. 9 and 9. The layout of the figures also corresponds to FIGS. 8 and 9.

FIGS. 12, 13 and 14 shoe the application of the method according to the invention for in vitro monitoring of the number of viable tumor cells (FIG. 12) and apoptotic/necrotic cells (FIG. 13) and the change in expression of the ICAM-1/HLA-ABC surface markers (FIG. 14) in the course of the therapy of a cancer patient.

On each of the first five days of the first week of therapy 2.5 mg dSLIM was administered to the patient with rectal carcinoma and metastases in the liver. On the sixth day of the first week radiation was carried out, followed by chemotherapy.

For the in vitro analysis of the in vivo effects, on each of the first sic days of the first week blood samples were taken from the patient. During the chemotherapy, blood samples were also taken towards the end of each week.

The plasma was isolated from the blood samples and incubated with cells of the tumor cell line HT-29. After this the number of viable cells (FIG. 12) and apoptotic/necrotic cells was determined, and the expression of the surface markers ICAM-1/HLA-ABC was investigated.

FIG. 12 shows the results of the incubation of HT-29 cells with plasma from eight blood samples. A clear reduction in the number of viable HT-29 cells can already be seen on the second day of dSLIM administration. The number of viable cells falls on the second day to less than half of the number of cells on the first day, which is comparable with the number of viable cells in the controls.

FIG. 13 shows the in vitro monitoring of apoptotic/necrotic tumor cells during the therapy of the cancer patient on days 1, 2, 5 and 20. In this evaluation of the monitoring of the in vivo effects it can be seen that one day after administration of dSLIM, the number of the apoptotic/necrotic cells is already clearly increasing.

FIG. 14 shows results from the investigations into the change in expression of the surface markers ICAM-1/HLA-ABC during the therapy of the cancer patient, using the plasma from the blood in samples 1, 2, 3 and 8. Here, sample 1 is used as a reference value for representing changes in the expression of the two surface markers.

On the second day of therapy ICAM-1 is already expressed much more strongly, which is visible in the lower section of the figure due to the shift in the position of the fluorescence intensity, which shows that ICAM-1 is more strongly expressed.

With HLA-ABC, on the second day still no shift of the fluorescence intensity has occurred. It does not take place until the third day of therapy and also shows stronger expression of HLA-ABC.

### LIST OF REFERENCE SIGNS

A=in vivo situation
B=in vitro immunoassay
1=Patient
2=Target tissue, e.g. tumor
3=Immune cells
4=Test substance, e.g. dSLIM
5=Activated immune cells
6=Donor
7=Immune cells, e.g. PBMC
8=Test substance, e.g. dSLIM
9=Activated immune cells, e.g. PBMC
10=Supernatant
11=Target cells, e.g. tumor cells
12=Analysis

1. A method for the in vitro investigation of the effect of substances in in vivo processes, comprising the following sequence of steps:
a) isolation of cells
b) primary incubation of the cells with the substance to be investigated
c) recovery of the supernatant or of the mixture of cells and supernatant from the primary incubation
d) secondary incubation of target cells with the supernatant or the mixture of cells and supernatant
e) analysis of the target cells.

2. The method according to claim 1, wherein the isolated cells for the primary incubation are effector cells of the immune system.

3. The method according to claim 1, wherein the target cells of the secondary incubation are human cells or cells from higher mammals.

4. The method according to claim 1, wherein the steps 1.d) and 1.e) are carried out using a patient's blood, serum, or plasma.

5. The method according to claim 1, wherein the substances to be investigated are immunomodulating and apoptosis-inducing or necrosis-inducing compounds.

6. An in vitro detection method that is suitable for the identification of immunomodulating compounds and/or the detection of the effect of immunomodulating compounds and the identification of apoptosis-inducing
and/or necrosis-inducing compounds mediated by the immune system in in vivo processes, comprising the following sequence of steps:
a) primary incubation of effector cells of the immune system with a substance to be investigated for immunomodulating effect or a substance inducing apoptosis or necrosis, followed by the
b) recovery of the supernatant or of the mixture of cells and supernatant from the primary incubation and followed by
c) secondary incubation of target cells with the supernatant or the mixture of cells and supernatant from the primary incubation, and finally
d) the immunomodulating and/or apoptosis-inducing and/or necrosis-inducing effect is analyzed by means of suitable detection methods.

7. The method according to claim 6, wherein the cells for the primary incubation are previously isolated in step 1.a.

8. The method according to claim 6, wherein the effector cells of the immune system for the primary incubation are preferably peripheral mononuclear cells from blood, spleen cells or subpopulations of cell mixtures sorted using FACS or MACS, such as for example B, T and NK cells, monocytes or dendritic cells.

9. The method according to claim 6, wherein the cells for the primary incubation and the target cells of the secondary incubation are human cells or cells from higher mammals.

10. The method according to claim 6, wherein the steps 6.c) and 6.d) are carried out with a patient's blood, serum, or plasma.

11. The method according to claim 6, wherein the target cells of the secondary incubation are tumor cells or cell lines genetically descended from a tumor.

12. The method according to claim 6, wherein the immunomodulating compounds whose effect is investigated are CpG-containing oligodeoxynucleotides or partially double-stranded DNA constructs with at least one CpG motif in a single-strand region.

13. The method according to claim 6, whereby the apoptosis-inducing and/or necrosis-inducing compounds whose effect is investigated are preferably antisense oligodeoxynucleotides, siRNA, antibodies or chemotherapeutic agents.

14. The method according to claim 6, wherein whole blood, blood cells, blood cell subpopulations, blood serum or blood plasma are used as the substance to be investigated in the primary incubation before, during and/or after a treatment.

15. The method according to claim 14, wherein incubations take place in an incubator.

16. The method according to claim 6, wherein supernatants are recovered by centrifugation.

17. The method according to claim 6, wherein the expression of specific proteins is investigated for analysis of the target cells.

18. The method according to claim 6, whereby the expression of defined genes is investigated for analysis of the target cells.

19. The method according to claim 6, wherein the target cells are stained for analysis, in particular with annexin V or propidium iodide stains.

20. The method according to claim 6, wherein apoptosis and/or necrosis detection methods are carried out for analysis of the target cells.

21. The method according to claim 6, wherein cell cycle analyses are carried out.

22. The method according to claim 6, wherein antibodies or other competing substances are added to the primary incubation with the substance to be investigated.

23. A kit for carrying out an in vitro detection method suitable for the identification of immunomodulating compounds and/or the detection of the effect of immunomodulating compounds and the identification of apoptosis-inducing and/or necrosis-inducing compounds mediated by the immune system in in vivo processes
aliquots of effector cells of the immune system prepared for storage and
means of carrying out primary and secondary incubation and
for the detection of messenger substances that are released as a reaction of the incubation of the cells in the primary incubation with a compound to be investigated, multi-well plates with 24 to 96 wells, in which the surfaces of the wells are coated with an antibody and/or
for the investigation of changes in the expression of surface molecules due to an immune reaction induced by the compound to be investigated, means of carrying out an RT-PCR, for which the kit contains suitable primers for the multiplication of the mRNA of surface molecules, enzymes for the multiplication, and the required buffers and/or means of an FACS analysis, for which the kit contains suitable fluorescence marked antibodies that are directed against surface antigens, and in addition means of preparing the target cells, such as buffers and chemicals.

24. A kit for in vitro demonstration of the effect of immunomodulating compounds and the identification of apoptosis-inducing and/or necrosis-inducing compounds mediated by the immune system in in vivo processes before, during and/or after the administration of such compounds, exhibiting at least the following components:
aliquots of target cells prepared for storage, for incubation with a patient's blood, serum, or plasma
means of carrying out a secondary incubation and
for the detection of messenger substances that are released as a reaction of the incubation of the cells in the primary incubation with a compound to be investigated, multi-well plates with 24 to 96 wells, in which the surfaces of the wells are coated with an antibody and/or
for the investigation of changes in the expression of surface molecules due to an immune reaction induced by the compound to be investigated, means of carrying out an RT-PCR, for which the kit contains suitable primers for multiplication of the mRNA of surface molecules, enzymes for multiplication, and the required buffers and/or means of an FACS analysis, for which the kit contains suitable fluorescence marked antibodies that are directed against surface antigens, and in addition means of preparing the target cells, such as buffers and chemicals.

25. The kit according to claim 24, wherein the target cells contained are tumor cells or cell lines genetically descended from a tumor.

26. The kit according to claim 24, wherein the target cells contained are tumor cells or cell lines genetically descended from a tumor.
